(19) United States
(12) Patent Application Publication
(10) Pub. No.: US 2025/0161285 A1
(43) Pub. Date: May 22, 2025

(54) BACTERIAL BIOFILM INHIBITORS

(71) Applicant: THE RESEARCH INSTITUTE AT NATIONWIDE CHILDREN'S HOSPITAL, Columbus, OH (US)

(72) Inventors: John S. GUNN, Powell, OH (US); Jenna SANDALA, Pittsburgh, PA (US); Christian Corey MELANDER, Edwardsburg, MI (US); Katherine June WOOLARD

(21) Appl. No.: 18/840,485

(22) PCT Filed: Feb. 24, 2023
(86) PCT No.: PCT/US2023/013799

§ 371 Date: Aug. 21, 2024

Related U.S. Application Data
(60) Provisional application No. 63/313,642, filed on Feb. 24, 2022.

(51) Int. Cl. A61K 31/4535; A61K 31/47; A61P 31/04; C07D 409/12

(52) U.S. Cl. CPC A61K 31/4535; A61K 31/47; A61P 31/04; C07D 409/12

(57) ABSTRACT

Compounds according to formula I: wherein Ar is an aryl or heteroaryl group, A is a C₁-C₃ alkyl, Z and Y are independently C₁-C₃ alkylene, X is C₁-C₃ alkylene, and R¹-R⁵ are selected from —H, halogen, C₁-C₃ alkyl, or phenyl, or a pharmaceutically acceptable salt thereof, are described. The compounds can be used to decrease the amount of biofilm in a subject, and can be used together with antibacterial agents for the treatment of bacterial infection.

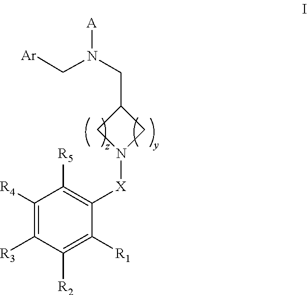

### CROSS-REFERENCE TO RELATED APPLICATIONS

This application claims priority to U.S. Provisional Application Ser. No. 63/313,642, filed on Feb. 24, 2022, which is hereby incorporated by reference in its entirety.

### GOVERNMENT FUNDING

This invention was made with government support under Grant No. AI116917 awarded by the National Institutes of Health. The government has certain rights in the invention.

### BACKGROUND

The Salmonella enterica subspecies cause an estimated 93 million infections globally every year (Majowicz et al., 2010. Clin. Infect. Dis. 50: 882-889). Salmonella subspecies are categorized as typhoidal or nontyphoidal. Typhoidal subspecies include Salmonella enterica serovar Typhi (S. Typhi), and infection by this serovar results in typhoid fever. Non-typhoidal subspecies include all those that do not cause typhoid fever, and instead typically cause salmonellosis. S. Typhi transmission is typically fecal-oral, and while incidences of typhoid fever are low in the United States and Europe, there is a high burden of disease in developing regions of Sub-Saharan Africa and Southeast Asia (Gunn et al., Trends Microbiol. 2014. 22(11): 648-655). In these areas, sanitation is frequently lacking and wastewater treatment is underdeveloped, and an estimated 14 million new infections and 136,000 deaths occur each year (GBD 2017 Typhoid and Paratyphoid Collaborators, 2019. Lancet Infect Dis. 19(4): 369-381).

While treatment with fluoroquinolone antibiotics, such as ciprofloxacin, is typically successful in clearing the infection, about 3-5% of acutely-infected patients are estimated to develop chronic S. Typhi infections due to biofilm colonization in the gallbladder, leading to fecal shedding of bacteria even after resolution of symptoms (Majowicz et al., 2010. Clin. Infect. Dis. 50: 882-889; Crawford et al., 2010. Proc Natl Acad Sci USA. 107(9): 4353-4358). Humans are the only known reservoir for typhoidal serovars of Salmonella, and this fecal shedding drives re-infection of water sources in areas with poor sanitation, and perpetuates the disease in these populations. In the gallbladder, the bacteria establish and maintain infection by forming biofilms on the surface of gallstones, which are present in up to 90% of chronic carriers (Flemming et al., 2010. Nat Rev Microbiol. 8(9): 623-633). Biofilms are defined as a surface-attached community of bacteria encased in an extracellular matrix of biomolecules (Flemming et al., 2016. Nat. Rev. Microbiol. 14: 563-575). The biofilm confers inherent tolerance that allows Salmonella bacteria to survive harsh environments, such as bile within the gallbladder, as well as host immune responses and antibiotic treatment. It has been shown that Salmonella biofilms can be up to 1000-fold more resistant to antibiotic treatment than planktonic Salmonella (Huggins et al., 2018. Med. Chem. Commun. 9: 1547-1552). When a patient develops a chronic S. Typhi infection, antibiotic treatment is only moderately successful, necessitating expensive, invasive methods such as cholecystectomy to clear chronic carriage (Thaver et al., 2009. BMJ, 338, b1865).

Given the importance of biofilms in chronic carriage of S. Typhi infections and the spread of typhoid fever in endemic regions, anti-biofilm treatments represent a promising potential strategy to reduce the spread of infection. Small molecules, such as 2-aminoimidazoles, 2-aminobenzimidazoles, furanones, and N-acyl homoserine lactone derivatives, have previously been used to inhibit biofilm formation and disrupt preformed biofilms of many pathogens (Huggins et al., 2018. Med. Chem. Commun. 9: 1547-1552; Wang et al., 2014. Curr Med Chem. 21(3): 296-311; Oppenheimer-Shaanan et al., 2013. Trends Microbiol. 21(11): 594-601; Pan et al., 2015. J Appl Microbiol. 119(5): 1403-1411; and Weig et al., RSC Medicinal Chemistry. 2021, Advance Article “A Scaffold Hopping Strategy to Generate New Aryl-2-Amino Pyrimidine MRSA Biofilm Inhibitors”). The inventors recently reported the identification of compound 1 from a screen of 4000 small molecules for Salmonella biofilm inhibition using S. Typhimurium as a surrogate for S. Typhi biofilms (Sandala et al., 2020. PLoS Pathog. 16(12): e1009192). Compound 1 also disrupts pre-formed biofilms in vitro, an effect that was enhanced upon combination with ciprofloxacin. In a murine model of chronic gallbladder Salmonella carriage, the combination of compound 1 (10 mg/kg/day) and ciprofloxacin (1.0 mg/kg/day) effected a 3-4.5 log reduction in the bacterial burden in the gallbladder, without concomitant bacterial dissemination to peripheral organs, indicating the potential of a dual therapy approach to the clearance of chronic Salmonella carriage in the gallbladder (Sandala et al., 2020. PLoS Pathog. 16(12): e1009192).

### SUMMARY OF THE INVENTION

Salmonella enterica serovars cause millions of infections each year that result either in typhoid fever or salmonellosis. Among those serovars that cause typhoid fever, Salmonella enterica subspecies Typhi can form biofilms on gallstones in the gallbladders of acutely-infected patients, leading to chronic carriage of the bacterium. These biofilms are recalcitrant to antibiotic-mediated eradication, leading to chronic fecal shedding of the bacteria, which results in further disease transmission. Herein, it is reported the synthesis and anti-biofilm activity of a 55-member library of small molecules based upon a previously identified hit that both inhibits and disrupts S. Typhi and S. Typhimurium (a nontyphoidal model serovar for S. Typhi) biofilms. Lead compounds inhibit S. Typhimurium biofilm formation in vitro at sub-micromolar concentrations, and disperse biofilms with five-fold greater potency than the parent compound. Three of the most promising compounds demonstrated synergy with ciprofloxacin in a murine model of chronic Salmonella carriage. This work furthers the development of effective anti-biofilm agents as a promising therapeutic avenue for the eradication of typhoidal Salmonella.

### BRIEF DESCRIPTION OF THE FIGURES

The present invention may be more readily understood by reference to the following drawings wherein:

FIGS. 1A and 1B provide A) an image of Compound 1 divided into tail (green), core (blue), and head (red) sections; and B) a scheme showing the general structure-activity analysis and testing approach used by the inventors.

FIG. 2 provides a scheme showing a second set of compounds 6a-n.

FIG. 3 provides a scheme showing a third set of compounds 7a-u.

FIG. 4 provides a scheme showing a fourth set of compounds 8a-k.

FIGS. 5A-5F provide graphs showing Enumeration of Salmonella in the gallbladder, liver, and spleen of infected mice at 15 dpi. 129X1/SvJ mice were fed a lithogenic diet for 8 wks. prior to intraperitoneal (I.P.) infection with 103 S. Typhimurium. In panels A-C, mice were administered I.P. a vehicle control (DMSO) or one of four combination treatments consisting of 1 mg/kg/day ciprofloxacin (cipro)+5 mg/kg/day compound 1, 7b, 7d, or 8j from days 5-15 post-infection. In panels D-F, mice were administered I.P. a vehicle control, 2 mg/kg/day cipro alone, 4 mg/kg/day cipro alone, 5 mg/kg/day 7d+2 mg/kg/day cipro, or 5 mg/kg/day 7d+4 mg/kg/day cipro from days 5-15 post-infection. Dotted lines represent the limit of detection where applicable; significant differences among the bacterial burden of treatment groups were determined via one-way ANOVA with the Tukey correction for multiple comparisons; ns non-significant, * p<0.05, ** p<0.01, *** p<0.001, **** p<0.0001.

FIG. 6 provides a scheme showing a fifth set of compounds 3.1a-1.

FIG. 7 provides a scheme showing a sixth set of compounds 3.2a-i.

FIG. 8 provides a scheme showing a seventh set of compounds 3.4a-g.

FIG. 9 provides a scheme showing an eight set of compounds 3.8a-j.

### DETAILED DESCRIPTION OF THE INVENTION

The present invention provides a compound according to formula I:

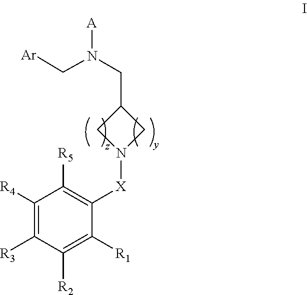

wherein Ar is an aryl or heteroaryl group, A is a C₁-C₃ alkyl, Z and Y are independently C₁-C₃ alkylene, X is C₁-C₃ alkylene, and R¹-R⁵ are selected from —H, halogen, C₁-C₃ alkyl, or phenyl, or a pharmaceutically acceptable salt thereof. The present invention also provides a method of decreasing the amount of biofilm in a subject, or for the treatment of bacterial infection.

### Definitions

The terminology as set forth herein is for description of the embodiments only and should not be construed as limiting of the invention as a whole. As used in the description of the invention and the appended claims, the singular forms “a”, “an”, and “the” are inclusive of their plural forms, unless contraindicated by the context surrounding such.

As used herein, the term “organic group” is used to mean a hydrocarbon group that is classified as an aliphatic group, cyclic group, or combination of aliphatic and cyclic groups (e.g., alkaryl and aralkyl groups). In the context of the present invention, suitable organic groups for the compounds of this invention are those that do not interfere with the anti-biofilm activity of the compounds. In the context of the present invention, the term “aliphatic group” means a saturated or unsaturated linear or branched hydrocarbon group. This term is used to encompass alkyl, alkenyl, and alkynyl groups, for example.

As used herein, the terms “alkyl”, “alkenyl”, and the prefix “alk-” are inclusive of straight chain groups and branched chain groups. Unless otherwise specified, these groups contain from 1 to 20 carbon atoms, with alkenyl groups containing from 2 to 20 carbon atoms. In some embodiments, these groups have a total of at most 10 carbon atoms, at most 8 carbon atoms, at most 6 carbon atoms, or at most 4 carbon atoms. Alkyl groups including 4 or fewer carbon atoms can also be referred to as lower alkyl groups. Alkyl groups can also be referred to by the number of carbon atoms that they include (i.e., C₁-C₄ alkyl groups are alky groups including 1-4 carbon atoms).

Cycloalkyl, as used herein, refers to an alkyl group (i.e., an alkyl, alkenyl, or alkynyl group) that forms a ring structure. Cyclic groups can be monocyclic or polycyclic and preferably have from 3 to 10 ring carbon atoms. A cycloalkyl group can be attached to the main structure via an alkyl group including 4 or less carbon atoms. Exemplary cyclic groups include cyclopropyl, cyclopropylmethyl, cyclopentyl, cyclohexyl, adamantyl, and substituted and unsubstituted bornyl, norbornyl, and norbornenyl.

Unless otherwise specified, “alkylene” and “alkenylene” are the divalent forms of the “alkyl” and “alkenyl” groups defined above. The terms, “alkylenyl” and “alkenylenyl” are used when “alkylene” and “alkenylene”, respectively, are substituted. For example, an arylalkylenyl group comprises an alkylene moiety to which an aryl group is attached.

The term “haloalkyl” is inclusive of groups that are substituted by one or more halogen atoms, including perfluorinated groups. This is also true of other groups that include the prefix “halo-”. Examples of suitable haloalkyl groups are chloromethyl, trifluoromethyl, and the like. Halo moieties include chlorine, bromine, fluorine, and iodine.

The term “aryl” as used herein includes single aromatic rings or multiring systems. Examples of aryl groups include phenyl, naphthyl, biphenyl, fluorenyl and indenyl. Aryl groups may be substituted or unsubstituted.

Unless otherwise indicated, the term “heteroatom” refers to the atoms O, S, or N. The term “heteroaryl” includes aromatic rings or ring systems that contain at least one ring heteroatom (e.g., O, S, N). In some embodiments, the term “heteroaryl” includes a ring or ring system that contains 2 to 12 carbon atoms, 1 to 3 rings, 1 to 4 heteroatoms, and O, S, and/or N as the heteroatoms. Suitable heteroaryl groups include furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, pyrazinyl, 1-oxidopyridyl, pyridazinyl, triazinyl, tetrazinyl, oxadiazolyl, thiadiazolyl, and so on.

The terms “arylene” and “heteroarylene” are the divalent forms of the “aryl” and “heteroaryl” groups defined above. The terms “arylenyl” and “heteroarylenyl” are used when “arylene” and “heteroarylene”, respectively, are substituted. For example, an alkylarylenyl group comprises an arylene moiety to which an alkyl group is attached.

Unless otherwise indicated, the term “heteroatom” refers to the atoms O, S, or N. The term “heteroaryl” includes aromatic rings or ring systems that contain at least one ring heteroatom (e.g., O, S, N). In some embodiments, the term “heteroaryl” includes a ring or ring system that contains 2 to 12 carbon atoms, 1 to 3 rings, 1 to 4 heteroatoms, and O, S, and/or N as the heteroatoms. Suitable heteroaryl groups include furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, pyrazinyl, 1-oxidopyridyl, pyridazinyl, triazinyl, tetrazinyl, oxadiazolyl, thiadiazolyl, and so on.

When a group is present more than once in any formula or scheme described herein, each group (or substituent) is independently selected, whether explicitly stated or not. For example, for the formula —C(O)—NR₂ each R group is independently selected.

As a means of simplifying the discussion and the recitation of certain terminology used throughout this application, the terms “group” and “moiety” are used to differentiate between chemical species that allow for substitution or that may be substituted and those that do not so allow for substitution or may not be so substituted. Thus, when the term “group” is used to describe a chemical substituent, the described chemical material includes the unsubstituted group and that group with nonperoxidic O, N, S, Si, or F atoms, for example, in the chain as well as carbonyl groups or other conventional substituents. Where the term “moiety” is used to describe a chemical compound or substituent, only an unsubstituted chemical material is intended to be included. For example, the phrase “alkyl group” is intended to include not only pure open chain saturated hydrocarbon alkyl substituents, such as methyl, ethyl, propyl, tert-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ether groups, haloalkyls, nitroalkyls, carboxyalkyls, hydroxyalkyls, cyanoalkyls, etc. On the other hand, the phrase “alkyl moiety” is limited to the inclusion of only pure open chain saturated hydrocarbon alkyl substituents, such as methyl, ethyl, propyl, tert-butyl, and the like.

A subject, as defined herein, is an animal such as a vertebrate or invertebrate organism. In other embodiments, the subject is a mammal such as a domesticated farm animal (e.g., cow, horse, pig) or pet (e.g., dog, cat). More preferably, the subject is a human.

“Treat”, “treating”, and “treatment”, etc., as used herein, refer to any action providing a benefit to a subject at risk for or afflicted with a condition or disease such as bacterial infection, including improvement in the condition through lessening or suppression of at least one symptom, delay in progression of the disease, prevention or delay in the onset of the disease, etc.

“Preventing,” as used herein, refers to any action that decreases the risk that a subject will develop an infection, or that will decrease the risk of symptoms should an infection nonetheless occur. Preventing infection can be done in subjects who have an increased risk of developing an infection. Subjects can have an increased risk of developing an infection as a result of, for example, being immunosuppressed or having recently been exposed to other individuals who are infected.

“Pharmaceutically acceptable” as used herein means that the compound or composition is suitable for administration to a subject for the methods described herein, without unduly deleterious side effects in light of the severity of the disease and necessity of the treatment.

The terms “therapeutically effective” and “pharmacologically effective” are intended to qualify the amount of each agent which will achieve the goal of decreasing disease severity while avoiding adverse side effects such as those typically associated with alternative therapies. The therapeutically effective amount may be administered in one or more doses.

### Anti-Biofilm Compounds

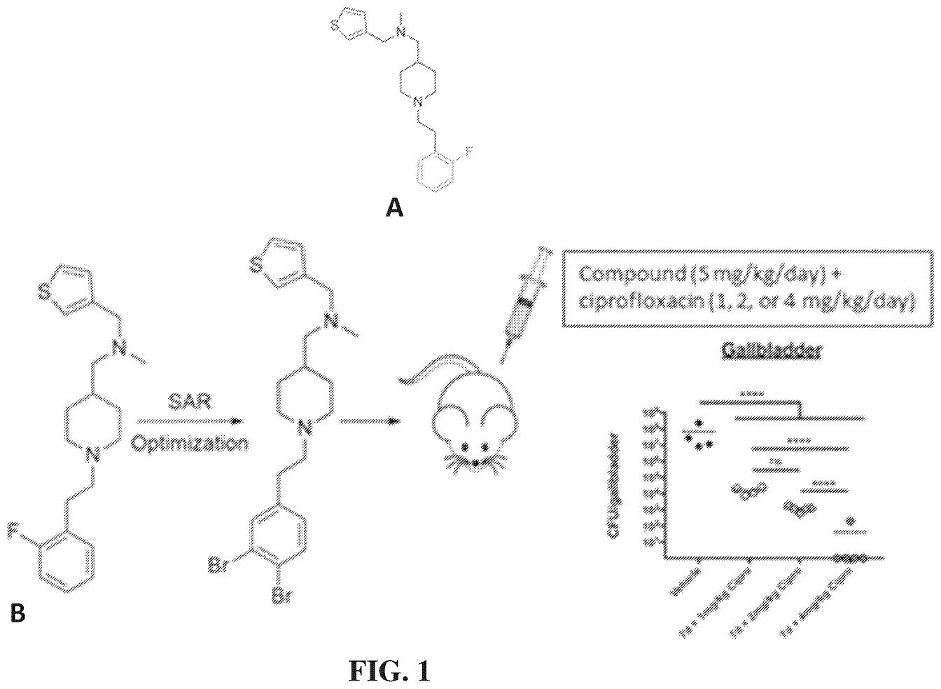

In one aspect, the present invention provides a compound according to formula I:

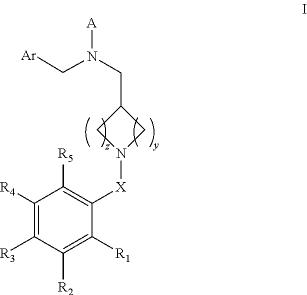

wherein Ar is an aryl or heteroaryl group, A is a C₁-C₃ alkyl, Z and Y are independently C₁-C₃ alkylene, X is C₁-C₃ alkylene, and R¹-R⁵ are selected from —H, halogen, C₁-C₃ alkyl, or phenyl, or a pharmaceutically acceptable salt thereof. The compound of formula I includes a head, core, and tail region, as shown in FIG. 1.

The inventors have carried out structure-activity studies to evaluate the effect of varying the core structure of the compound of formula I on activity. For instance, Z and Y in the core region are defined by formula I as being independently C₁-C₃ alkylene. For instance, in some embodiments, Z and Y are the same. If Z and Y are the same and both C₂ alkylene, the core structure comprises a piperidine ring, whereas if Z and Y are the same and both C1 the core structure comprises an azetidine ring.

The group X can be a C₁ alkyl (i.e., methylene) group, a C₂ alkyl (i.e., ethylene) group, or a C₃ alkyl (i.e., propylene) group. In some embodiments, X is C₂ alkylene, as shown in Formula II:

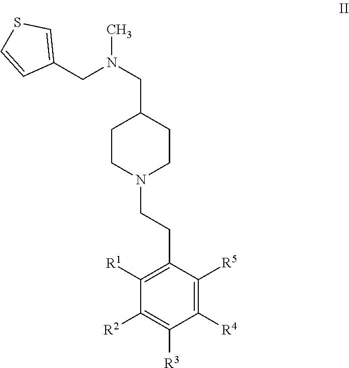

In some embodiments, one or two of R¹-R⁵ are selected from bromine, chlorine, iodine, or phenyl, and the remainder of R¹-R⁵ are hydrogen. The inventors have determined that many compounds including chlorine, bromine, or iodine moieties on the tail phenyl ring exhibit increased activity. In some embodiments, two of R¹-R⁵ are chlorine, bromine, or iodine, and the remaining R groups are —H. In further embodiments, R¹ and R² are chlorine, R¹ and R³ are chlorine, or R² and R³ are bromine, and the remaining R groups are —H. In some embodiments, R³ is phenyl and R¹, R², R⁴, and R⁵ are —H.

The inventors have tested a variety of different aryl or heteroaryl group for the head region of the compound (the Aromatic group (Ar)), as described in Example 2 herein. In some embodiments, Ar is an aryl group. In further embodiments, Ar is an aromatic five-membered ring, such as a thiophene group. In yet further embodiments, Ar is a substituted or unsubstituted phenyl group. For example, in some embodiments, Ar is a halogenated phenyl group.

The inventors determined that a number of compounds were particularly effective. Biphenyl compound 8j exhibited the highest dispersion activity, compound 7b demonstrated the most potent inhibition activity, while 7d displayed the lowest combined IC₅₀ and EC₅₀ values. The structures of these compounds are shown below:

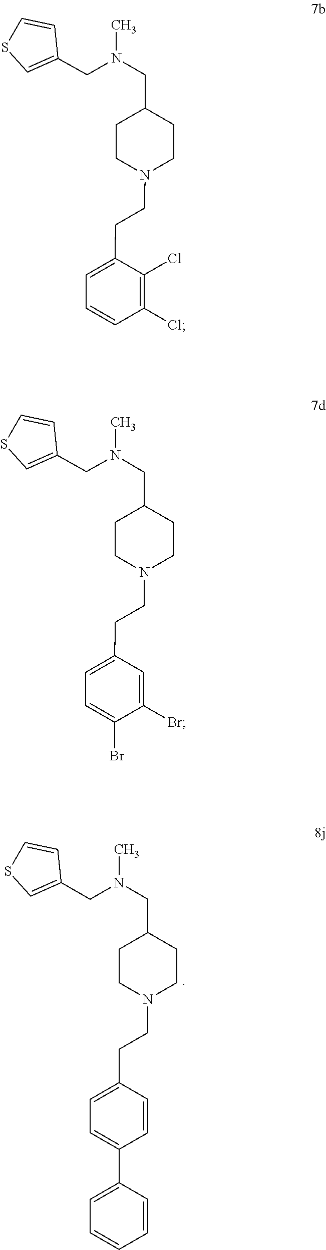 [SMILES: CN(Cc1ccsc1)CC1CCN(CCc2cccc(Cl)c2Cl)CC1 ; CN(Cc1ccsc1)CC1CCN(CCc2ccc(Br)c(Br)c2)CC1 ; CN(Cc1ccsc1)CC1CCN(CCc2ccc(-c3ccccc3)cc2)CC1]

The invention is inclusive of the compounds described herein in any of their pharmaceutically acceptable forms, including, tautomers, salts, solvates, polymorphs, prodrugs, and the like. In particular, if a compound isoptically active, the invention specifically includes each of the compound's enantiomers as well as racemic mixtures of the enantiomers. It should be understood that the term “compound” includes any or all of such forms, whether explicitly stated or not (although at times, “salts” are explicitly stated).

The compounds disclosed herein may contain one or more asymmetric carbon atoms, so that the compounds can exist in different stereoisomeric forms. The compounds can be, for example, racemates or optically active forms. The optically active forms can be obtained by resolution of the racemates or by asymmetric synthesis. In some instances, the compounds disclosed herein are R enantiomers. In other instances, the compounds disclosed herein are S enantiomers. In some instances, the compounds disclosed herein are varying mixtures of enantiomers.

### Methods of Decreasing Biofilm

Another aspect of the invention provides a method of decreasing bacterial biofilm in a subject, comprising administering a therapeutically effective amount of a compound according to formula I:

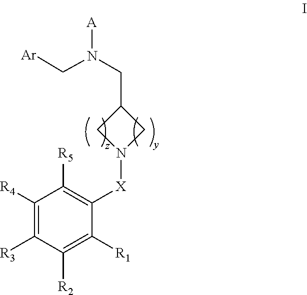

to the subject; wherein Ar is an aryl or heteroaryl group, A is a C₁-C₃ alkyl, Z and Y are independently C₁-C₃ alkylene, X is C₁-C₃ alkylene, and R¹-R⁵ are selected from —H, halogen, C₁-C₃ alkyl, or phenyl, or a pharmaceutically acceptable salt thereof.

The compounds used in the method include any of the compounds of formula I described herein. For example, in some embodiments, the aromatic group of formula I is a thiophene group. In further embodiments, one or two of R¹-R⁵ are selected from bromine, chlorine, iodine, or phenyl, and the remainder of R¹-R⁵ are hydrogen. In additional embodiments, X is C₂ alkylene, while in further embodiments two of R¹-R⁵ are chlorine, bromine, or iodine, and the remaining R groups are —H.

A biofilm is a microbial community that produces a slimy extracellular matrix composed of extracellular polymeric substances that can be formed on a living or non-living surface by bacteria, and serves to protect the bacteria within and provide other advantages such as allowing the bacteria to share nutrients.

Biofilms form in several stages. First, the bacteria accumulates on a surface. The bacteria then deposits a layer of molecules known as a conditioning film, after which the bacteria adhere to the conditioning film. Adhesion to a surface alters the phenotype of the bacterium, changing activities like respiration, oxygen uptake, electron transport, synthesis of extracellular polymers, etc. The bacteria then enter the colonization stage, during which the biofilm forms. In the colonization stage, the bacteria synthesizes extracellular matrix molecules and the number of attached bacteria is increased. These additional organisms may be the same or different species as the already-adhered cells. The colonized cells then continue to grow leading to the formation of dense bacterial aggregates.

A large number of bacteria are known to produce biofilms. Examples of pathogenic bacterial known to form biofilms include Enterococcus faecalis, Staphylococcus aureus, Staphylococcus epidermidis, Streptococcus viridans, E. coli, Salmonella enterica serovar Typhi, Salmonella enterica serovar Typhimurium, Klebsiella pneumoniae, Proteus mirabilis and Pseudomonas aeruginosa. In some embodiments, the biofilm is produced by Salmonella. In some instances, low-dose antibiotic administration can induce biofilm formation. Kaplan, J., Int J Artif Organs, 34(9):737-51 (2011).

The inventors have demonstrated that compounds of formula I are capable of breaking down bacterial biofilm. Accordingly, in some embodiments, administration of a compound according to formula I decreases the amount of bacterial biofilm in the subject. The amount of the decrease in the bacterial biofilm can range from at least a 10% decrease, a 20% decrease, a 30% decrease, a 40% decrease, a 50% decrease, a 60% decrease, a 70% decrease, an 80% decrease, a 90% decrease, or a 100% decrease (i.e., total elimination of the bacterial biofilm).

A subject infected by pathogenic bacteria can have biofilm form in a variety of different parts of the body. Common sites of biofilm formation include the skin, teeth, and mucosa. Medical implants can also result in biofilm formation on the medical implant. Biofilms facilitate colonization and persistent infection by Salmonella in gallbladders of humans and mouse models of chronic carriage. Adcox et al., Infect Immun., 84(11):3243-3251 (2016). Accordingly, in some embodiments, the method comprises decreasing bacterial biofilm in the gallbladder of the subject.

### Methods of Treating or Preventing Bacterial Infection

In another aspect, the present invention provides a method of treating or preventing bacterial infection in a subject, comprising administering a therapeutically effective amount of a compound according to formula I:

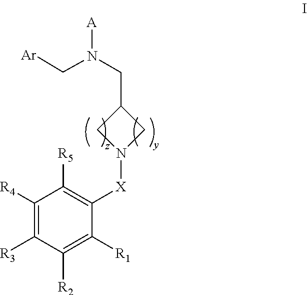

to the subject; wherein Ar is an aryl or heteroaryl group, A is a C₁-C₃ alkyl, Z and Y are independently C₁-C₃ alkylene, X is C₁-C₃ alkylene, and R¹-R⁵ are selected from —H, halogen, C₁-C₃alkyl, or phenyl, or a pharmaceutically acceptable salt thereof.

The compounds used in the method include any of the compounds of formula I described herein. For example, in some embodiments, one or two of R¹-R⁵ are selected from bromine, chlorine, iodine, or phenyl, and the remainder of R¹-R⁵ are hydrogen, while in further embodiments X is C₂ alkylene.

The present invention provides a method of treating or preventing bacterial infection in a subject. Bacterial infection refers to infection of the subject by pathogenic bacteria. The bacteria can be either gram-negative bacteria or gram-positive bacteria. A wide variety of pathogenic bacteria are known to those skilled in the art. Examples of pathogenic bacterial species include Mycobacterium tuberculosis, Bordella pertussis, Chlamydia trachomatis, Salmonella Typhi, Escherichia coli, Francisella tularensis, Helicobacter pylori, Vibrio cholerae, Clostridium botulinum, Streptococcus pneumoniae, Yersinia enterocolitica, and Staphylococcus aureus. In some embodiments, the bacterial infection is a Salmonella infection. In some embodiments, the pathogenic bacteria are bacteria capable of forming a biofilm. In some embodiments, only treatment is provided, while in other embodiments, administration of the compound is prophylactic. Preventive treatment can be administered to a subject who has an increased risk of developing a bacterial infection.

The compounds of Formula I can be administered together with an antibiotic compound (i.e., a second compound) to provide more effective treatment or prevention of bacterial infection. Suitable antibiotics include bactericidal or bacteriostatic compounds already known in the art. Examples of known antibiotics include agents that target the bacterial cell wall, such as penicillins, cephalosporins, agents that target the cell membrane such as polymyxins, agents that interfere with essential bacterial enzymes, such as quinolones and sulfonamides, and agents that that target protein synthesis such as the aminoglycosides, macrolides and tetracyclines. Additional known antibiotics include cyclic lipopeptides, glycylcyclines, and oxazolidinones. In some embodiments, the antibiotic is a fluoroquinolone compound such as ciprofloxacin.

### Formulation and Administration

The present invention provides a method for administering one or more anti-bacterial and/or anti-biofilm compounds together with a pharmaceutically acceptable carrier. Examples of pharmaceutical carriers or compositions include those for oral, intravenous, intramuscular, subcutaneous, or intraperitoneal administration, or any other route known to those skilled in the art, and generally involves providing a compound formulated together with a pharmaceutically acceptable carrier.

When preparing the compounds described herein for oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable carrier.

For intravenous, intramuscular, subcutaneous, or intraperitoneal administration, the compound may be combined with a sterile aqueous solution which is preferably isotonic with the blood of the recipient. Such formulations may be prepared by dissolving solid active ingredient in water containing physiologically compatible substances such as sodium chloride, glycine, and the like, and having a buffered pH compatible with physiological conditions to produce an aqueous solution, and rendering said solution sterile. The formulations may be present in unit or multi-dose containers such as sealed ampoules or vials.

Formulations suitable for parenteral administration conveniently comprise a sterile aqueous preparation of the active compound which is preferably made isotonic. Preparations for injections may also be formulated by suspending or emulsifying the compounds in non-aqueous solvent, such as vegetable oil, synthetic aliphatic acid glycerides, esters of higher aliphatic acids or propylene glycol.

The dosage form and amount can be readily established by reference to known treatment or prophylactic regiments. The amount of therapeutically active compound that is administered and the dosage regimen for treating a disease condition with the compounds and/or compositions of this invention depends on a variety of factors, including the age, weight, sex, and medical condition of the subject, the severity of the disease, the route and frequency of administration, and the particular compound employed, the location of the unwanted proliferating cells, as well as the pharmacokinetic properties of the individual treated, and thus may vary widely. The dosage will generally be lower if the compounds are administered locally rather than systemically, and for prevention rather than for treatment. Such treatments may be administered as often as necessary and for the period of time judged necessary by the treating physician. One of skill in the art will appreciate that the dosage regime or therapeutically effective amount of the inhibitor to be administrated may need to be optimized for each individual. The pharmaceutical compositions may contain active ingredient in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight, may be appropriate. The daily dose can be administered in one to four doses per day.

The anti-biofilm or anti-bacterial compounds can also be provided as pharmaceutically acceptable salts. The phrase “pharmaceutically acceptable salts” connotes salts commonly used to form alkali metal salts and to form addition salts of free acids or free bases. The nature of the salt is not critical, provided that it is pharmaceutically acceptable. Suitable pharmaceutically acceptable acid addition salts of the compounds may be prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, araliphatic, heterocyclic, carboxylic, and sulfonic classes of organic acids, examples of which include formic, acetic, propionic, succinic, glycolic, gluconic, lactic, malic, tartaric, citric, ascorbic, glucoronic, maleic, fumaric, pyruvic, aspartic, glutamic, benzoic, anthranilic, mesylic, salicylic, p-hydroxybenzoic, phenylacetic, mandelic, ambonic, pamoic, methanesulfonic, ethanesulfonic, benzenesulfonic, pantothenic, 2-hydroxyethanesulfonic, toluenesulfonic, sulfanilic, cyclohexylaminosulfonic, stearic, algenic, γ-hydroxybutyric, galactaric, and galacturonic acids. Suitable pharmaceutically acceptable base addition salts of the compounds described herein include metallic salts made from aluminum, calcium, lithium, magnesium, potassium, sodium, and zinc. Alternatively, organic salts made from N,N′-dibenzylethylenediamine, chloroprocaine, choline, diethanolamine, ethylenediamine, meglumine (N-methylglucamine) and procaine may be used form base addition salts of the compounds described herein. All of these salts may be prepared by conventional means from the corresponding compounds described herein by reacting, for example, the appropriate acid or base with the compound.

### Preparation of Anti-Biofilm Compounds

Compounds of the invention may be synthesized by synthetic routes that include processes analogous to those well known in the chemical arts, particularly in light of the description contained herein. Preparation of the compounds is also described in the Example herein. The starting materials are generally available from commercial sources such as Aldrich Chemicals (Milwaukee, Wisconsin, USA) or are readily prepared using methods well known to those skilled in the art (e.g., prepared by methods generally described in Louis F. Fieser and Mary Fieser, Reagents for Organic Synthesis, v. 1-19, Wiley, New York, (1967-1999 ed.) and similar texts known to those skilled in the art.

The present invention is illustrated by the following examples. It is to be understood that the particular examples, materials, amounts, and procedures are to be interpreted broadly in accordance with the scope and spirit of the invention as set forth herein.

### EXAMPLES

### Example 1: Development of Small Molecules that Work Cooperatively with Ciprofloxacin to Clear Salmonella Biofilms in a Chronic Gallbladder Carriage Model

A structure-activity relationship (SAR) study of compound 1 was initiated with the aim of augmenting both biofilm inhibition and biofilm dispersion activity. From an analog development standpoint, compound 1 can be divided into three sections: the fluorophenyl alkyl tail (green), the aminopiperidine core (blue), and the thiophene head group (red) (FIG. 1). Structure-activity relationship findings are reported herein, focusing on modulating the structure of the fluorophenyl tail and the length of the linker between the tail and the aminopiperidine core. A library of 55 derivatives was constructed and screened for in vitro activity against S. Typhimurium biofilms, leading to the identification of 31 compounds with increased inhibition activity, and 38 compounds with increased dispersion activity in comparison to compound 1. Three derivatives were then evaluated in combination with ciprofloxacin in a murine model of chronic S. Typhimurium gallbladder carriage, of which all three showed synergy with ciprofloxacin towards eliminating bacterial burden, with compound 7d demonstrating the greatest disruptive capabilities. This work furthers the development of effective anti-biofilm agents as a promising therapeutic avenue for the eradication of typhoidal Salmonella.

### Discussion and Results

The inventors first generated a library of derivatives designed to probe the impact halide identity, number, and position had upon activity. Specifically, this library comprised modifications to the tail group that involved substitution of different halogens in place of the fluorine, as well as incorporation of the fluorine at different positions on the ring. Additionally, as previous SAR studies on anti-biofilm scaffolds have identified 3,5-dihalogenated phenyl motifs as effective in combatting biofilm formation (Bunders et al., 2010. Bioorg. Med. Chem. Lett. 20(12): 3797-3800), the 3,5-difluoro motif was also investigated. A derivative with a methoxy group was synthesized to test the effects of an electron-donating group on the activity of the compound, and a control compound was also generated that lacked the aromatic tail completely. The synthetic approach to this first set of analogs is outlined in Scheme 1. The Boc-protected aminopiperidine core (2) was first alkylated to generate Boc-protected piperidines 3a-g. The Boc group of the resulting compounds was removed and the resulting amine alkylated with 3-(bromomethyl)thiophene to generate the eight target compounds 4a-h and compound 5. In addition, an analog of compound 1 lacking the thiophene head group (4h) was generated to determine the necessity of this moiety.

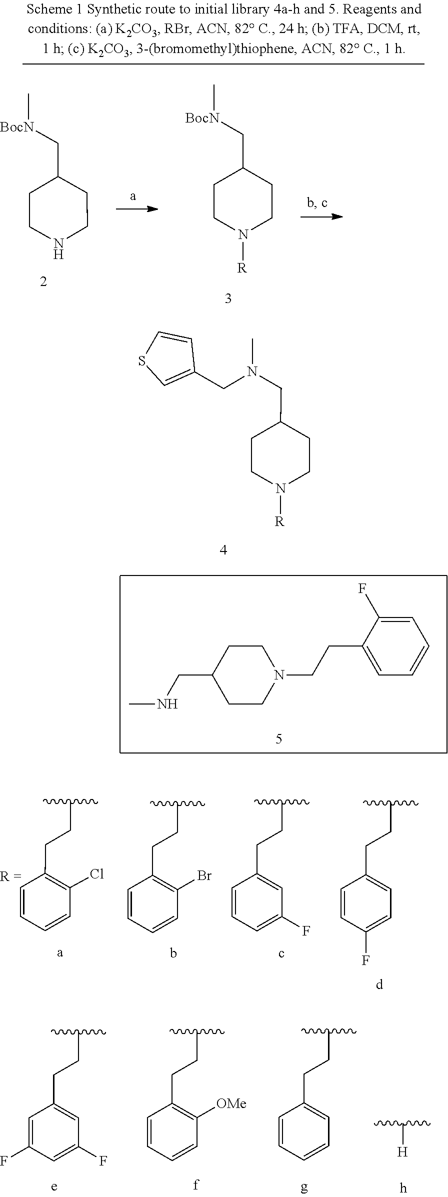

These nine compounds were tested for inhibition and dispersion of S. Typhimurium ATCC 14028 (JSG210) biofilms using a procedure that mimics growth in vivo (Weig et al., RSC Med Chem., 2020 Dec. 8; 12(2):293-296). Inhibition of biofilm formation is assessed by growing bacteria in a 96-well plate in the absence (control) or presence of test compounds. After 24 hours, the wells are washed to remove planktonic bacteria, and the remaining surface attached bacteria (biofilm) are stained with crystal violet (CV). The wells are then washed again to remove excess CV, and the remaining CV is solubilized with a solution of acetic acid and then quantified by spectrophotometry at 570 nm. A dose response curve is then constructed from which an IC₅₀ value is determined. Dispersion of established biofilms was quantified using a similar experimental approach with the exception that biofilms are established first over 24 hrs and then treated with compound. From the dose response curve, 50% dispersion of established biofilms can then be determined, which we refer to as EC₅₀ values.

Under these conditions, the parent compound returned an IC₅₀ of 5.7 μM and an EC₅₀ of 829 μM. The activity of the first library of analogs is summarized in Table 1. Of the compounds containing fluoro substituents (1, 4c-e), the most active biofilm inhibitor remained the original lead 1. The 4-fluoro derivative 4d was the most effective dispersion agent, returning an EC₅₀ of 235 μM (ca. 3.5 times more effective than 1). Interestingly, the 3,5-difluoro derivative 4e was inactive. Replacing the fluoro substituent with either a 2-chloro (4a) or 2-bromo (4b) derivative resulted in increased activity in terms of both inhibition and dispersion, with 2-chloro derivative 4a being the most active of these first analogs. Replacement of the fluoro substituent with a methoxy in 4f resulted in decreased inhibition and dispersion activity, as did complete removal of the halogens in 4g. Removal of the thiophene head (5) abrogated activity while removal of the tail (4h), had a significant detrimental impact on inhibition but interestingly led to a modest increase in dispersion activity.

TABLE 1
IC₅₀ and EC₅₀ values for compounds 1, 4a-h, and
5. All values are in μM and are presented
as the mean ± the standard deviation.

| Compound | IC₅₀ (μM) | EC₅₀ (μM) |
|---|---|---|
| 1 | 5.7 ± 0.9 | 829 ± 43 |
| 4a | 6.0 ± 1.4 | 232 ± 6 |
| 4b | 7.2 ± 1.6 | 308 ± 11 |
| 4c | 6.9 ± 0.1 | 388 ± 19 |
| 4d | 16.8 ± 3.4 | 235 ± 18 |
| 4e | >200^{a} | >12000 |
| 4f | 16.9 ± 5.2 | 11763 ± 77 |
| 4g | 21.2 ± 2.6 | >12000 |
| 4h | 138.1^{b} ± 8.6 | 442 ± 38 |
| 5 | >200^{b} | >12000 |

^{a}Growth inhibition at ≥50 μM;
^{b}Growth inhibition at ≥100 μM

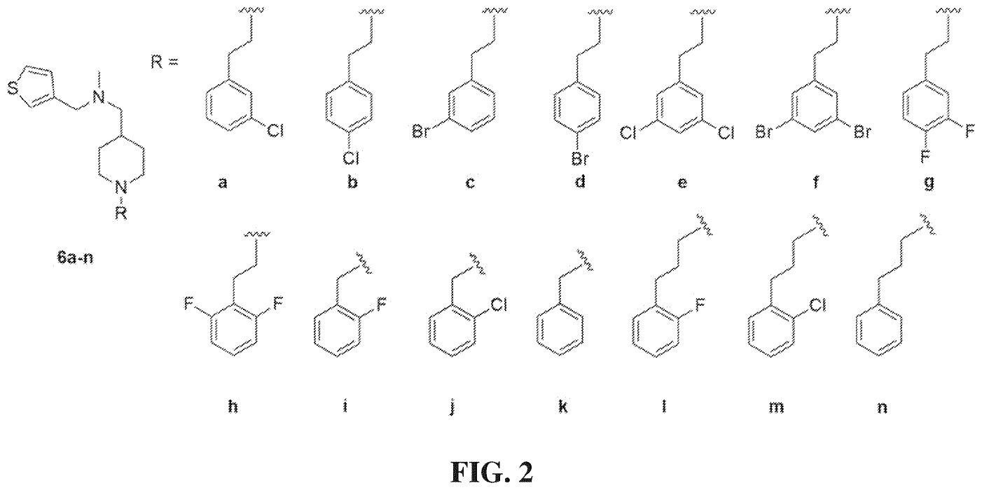

Leveraging this preliminary information, the next set of compounds included incorporation of chlorine and bromine at the 3- and 4-positions (6a-d) to further determine if there was a correlation between activity and halogen identity (FIG. 2). Compounds containing 3,4, 3,5, and 2,6 halogen substitution patterns (6e-h) were synthesized to probe whether di-halogenation in general was detrimental to activity. Lastly, the length of the linker between the tail and the piperidine core was both increased and decreased by one methylene unit to determine if this had any effect on activity. The activities of compounds 6a-n are summarized in Table 2.

TABLE 2
IC₅₀ and EC₅₀ values for compounds 6a-n. All
values are in μM compound and are presented
as the mean ± the standard deviation.

| Compound | IC₅₀ (μM) | EC₅₀ (μM) |
|---|---|---|
| 6a | 2.0^{a} ± 1.2 | 135 ± 12 |
| 6b | 3.0 ± 1.4 | 69 ± 4 |
| 6c | 3.6^{a} ± 0.9 | 83 ± 6 |
| 6d | 3.0 ± 0.7 | 57 ± 4 |
| 6e | 1.6^{b} ± 0.5 | 45 ± 2 |
| 6f | 3.4^{a} ± 1.4 | 42 ± 3 |
| 6g | 3.0 ± 0.2 | 87 ± 13 |
| 6h | 6.3 ± 0.6 | 138 ± 18 |
| 6i | 8.8 ± 0.8 | 125 ± 6 |
| 6j | 2.7 ± 0.7 | 1810 ± 176 |
| 6k | 26.7 ± 1.2 | >12000 |
| 6l | 11.5 ± 2.7 | 221 ± 17 |
| 6m | 5.4 ± 1.2 | 59 ± 4 |
| 6n | 78.1 ± 3.7 | >12000 |

^{a}Growth inhibition at ≥100 μM;
^{b}Growth inhibition at ≥50 μM

Following the trend observed with the first set of analogs, replacement of fluorine with either a chlorine or a bromine increased activity in the context of both biofilm inhibition and dispersion. However, while incorporation of chlorine in the 3-position resulted in an improved IC₅₀ of over two-fold (6a vs 4c), and in the 4-position of over four-fold (6b vs 4d), bromine incorporation had a larger effect on dispersion activity (6b vs 6d). Placing two fluorine atoms on the tail in positions 3 and 4 (6g) reduced both the IC₅₀ and EC₅₀ values by over two-fold compared to compounds 4c and 4d, indicating that these positions warranted further exploration via both chlorination and bromination. Strategic placement of chlorines at two positions on the ring as in 6e resulted in the highest activity for inhibition and second-highest for dispersion (1.6 μM/45 μM), while the bis-bromo analog 6f returned the lowest EC₅₀ value (42 μM). Decreasing the length of the linker by one methylene unit (6i-j) resulted in a decrease in IC₅₀ values and notably, a decrease in EC₅₀ values. Lengthening the linker by one methylene unit (6l-n), however, resulted in reduced activity for both inhibition and dispersion.

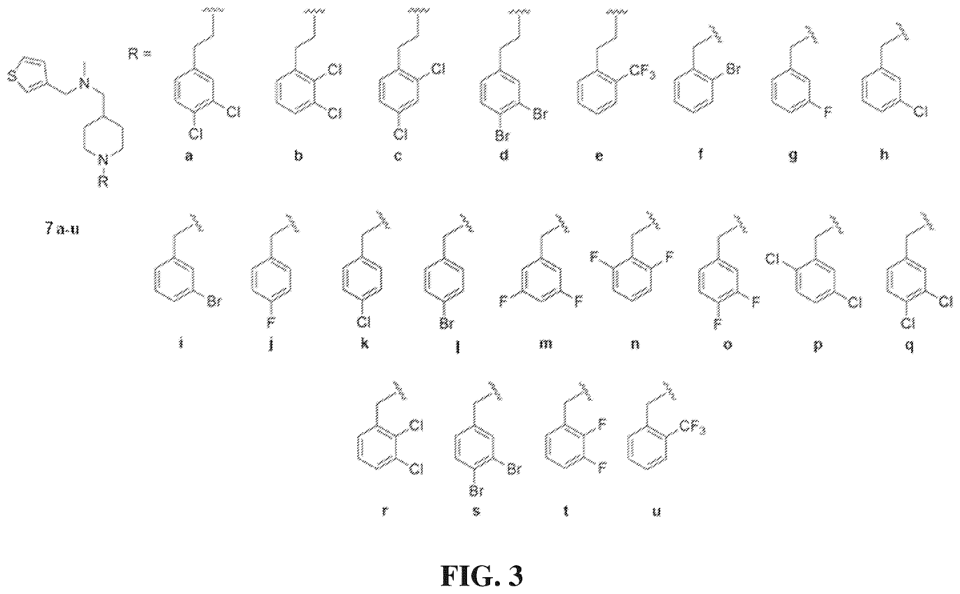

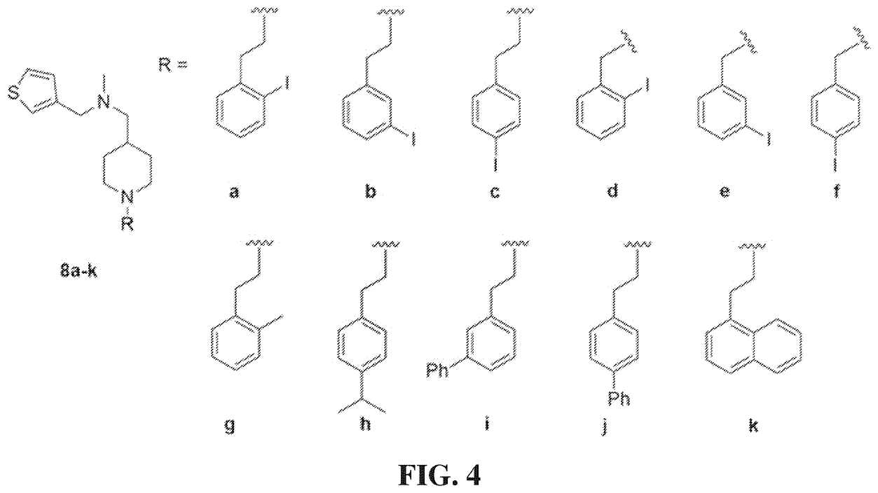

The final two libraries in this study focused on exploring the addition of multiple C₁/Br substituents on the tail, as well as tails connected via contracted or elongated linkers (FIG. 3). Additionally, six iodo derivatives were also synthesized (8a-f) to investigate the effects of a larger, less-electronegative halogen on activity (FIG. 4). Finally, five derivatives (8g-k) were generated as steric isosteres of chlorine, bromine and iodine to probe the effects of sterics versus electronics (FIG. 4). The activities of these compounds are reported together in Table 3.

TABLE 3
IC₅₀ and EC₅₀ values for compounds 7a-u and 8a-
k. All values are in μM and are presented
as the mean ± the standard deviation.

| Compound | IC₅₀ (μM) | EC₅₀ (μM) |
|---|---|---|
| 7a | 1.4^{a} ± 0.9 | 45 ± 2 |
| 7b | 0.49 ± 0.4 | 37 ± 4 |
| 7c | 2.5 ± 0.5 | 22 ± 2 |
| 7d | 1.7^{b} ± 0.1 | 25 ± 6 |
| 7e | 12.7 ± 4.1 | 60 ± 2 |
| 7f | 4.9 ± 0.8 | 79 ± 3 |
| 7g | 8.7 ± 1.3 | 1051 ± 77 |
| 7h | 5.2 ± 1.3 | 299 ± 21 |
| 7i | 6.5 ± 2.9 | 142 ± 9 |
| 7j | 12.9 ± 1.4 | 443 ± 52 |
| 7k | 5.9 ± 2.8 | 934 ± 26 |
| 7l | 8.5 ± 1.8 | 127 ± 13 |
| 7m | 14.2 ± 1.7 | >12000 |
| 7n | >200^{c} | >12000 |
| 7o | 7.2 ± 1.8 | 3803 ± 204 |
| 7p | 15.8 ± 1.4 | 196 ± 27 |
| 7g | 3.9 ± 1.2 | 55 ± 8 |
| 7r | 2.7 ± 1.4 | 70 ± 8 |
| 7s | 11.9 ± 1.7 | 32 ± 1 |
| 7t | 145.7^{c} ± 18.3 | 870 ± 79 |
| 7u | 20.9^{c} ± 6.1 | 140 ± 39 |
| 8a | 4.3^{a} ± 1.8 | 60 ± 2 |
| 8b | 4.0 ± 0.7 | 44 ± 2 |
| 8c | 2.6^{a} ± 0.4 | 89 ± 10 |
| 8d | 5.4 ± 0.5 | 79 ± 5 |
| 8e | 10.0 ± 4.5 | 92 ± 3 |
| 8f | 5.7 ± 2.9 | 77 ± 3 |
| 8g | 6.3 ± 2.3 | 247 ± 11 |
| 8h | 2.3^{b} ± 0.6 | 123 ± 7 |
| 8i | 1.8^{b} ± 0.4 | 31 ± 4 |
| 8j | 3.2^{b} ± 0.3 | 16 ± 1 |
| 8k | 2.5^{a} ± 0.8 | 28 ± 5 |

^{a}Growth inhibition at ≥50 μM;
^{b}Growth inhibition at ≥12.5 μM;
^{c}Growth inhibition at ≥100 μM

The data for these compounds revealed that while compounds with shorter linkers were generally more active than parent compound 1 for both inhibition and dispersion of biofilms, they were outperformed by several compounds with two methylene units between the halobenzene and the core. Notably compound 7b returned an IC₅₀ of 490 nM, making it, to the best of our knowledge, one of the most potent inhibitors of Salmonella biofilms reported to date. Additionally, compounds that contained steric isosteres of the various halogens proved to be more potent than the corresponding halogenated compounds. Notably, this was seen with the iodinated derivatives and the biphenyl compounds; 8i was two-fold more active at inhibiting biofilm formation than its iodinated counterpart 8b. A number of compounds also exhibited improved dispersion activity, including 7c, 7d, and 8j.

Based upon this in vitro data, compound 1 and three analogs from this study were selected for in vivo studies in a murine model of gallbladder carriage. Biphenyl compound 8j exhibited the highest dispersion activity with an EC₅₀ of 16 μM. Compound 7b demonstrated the most potent inhibition activity with an IC₅₀ of 490 nM. Lastly, 7d was selected due to its lowest combined IC₅₀ and EC₅₀ values of 1.7 μM and 25 μM, respectively. 129X1/SvJ NRAMP^{+/+} mice were fed a lithogenic diet for eight weeks in order to induce gallstone formation, thereby mimicking human carriers and allowing biofilm growth within the gallbladder following infection with S. Typhimurium. Mice were randomized to one of five 10-day treatment regimens: vehicle control (DMSO), 5 mg/kg/day compound 1+1 mg/kg/day ciprofloxacin (cipro), 5 mg/kg/day 7b+1 mg/kg/day cipro, 5 mg/kg/day 7d+1 mg/kg/day cipro, or 5 mg/kg/day 8j+1 mg/kg/day cipro. A dose of 5 mg/kg/day compound was chosen because previous experiments in mice treated with compound 1 at a dose of 10 mg/kg/day reduced bacterial burden in the gallbladder by a factor of several logs, nearing the limit of detection; thus, a lower dose was used in order to more precisely compare compound activities (Sandala et al., 2020. PLoS Pathog. 16(12): e1009192). Similarly, compounds were dosed in combination with cipro in order to prevent dissemination and accumulation of released bacteria in distal organs such as the liver and spleen, which we have previously observed when administering anti-biofilm compounds without concomitant antibiotics (Sandala et al., 2020. PLoS Pathog. 16(12): e1009192). An initial dose of 1 mg/kg/day cipro was chosen as we have previously shown that treating with cipro alone at this concentration was able to significantly reduce gallbladder-borne Salmonella in a model of acute infection (i.e., when gallbladder Salmonella are predominately planktonic), but not in a chronic model of infection (i.e., when gallbladder Salmonella exist primarily within a biofilm) (Sandala et al., 2020. PLoS Pathog. 16(12): e1009192; Gonzalez et al., 2018. Sci Rep. 8(1): 222).

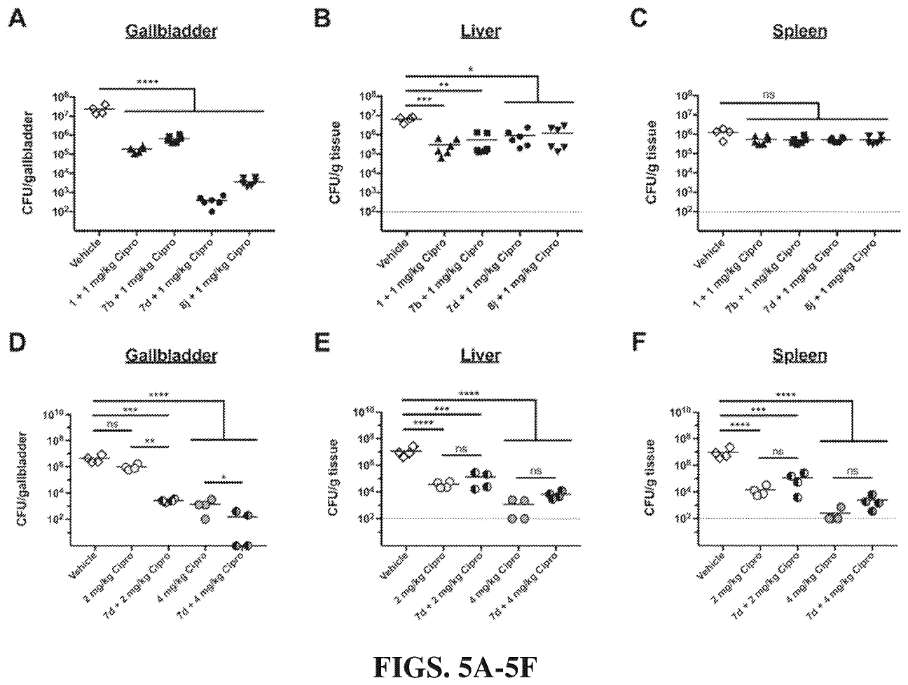

Compounds 7d and 8j effected the greatest reduction in bacterial burden in the gallblader (4.5-5 and 3.5-4 log-fold respectively). Compounds 1 and 7b, which were less active biofilm dispersing agents in vitro than 7d and 8j, were also less active in vivo (˜2 and ˜1.5 log-fold reduction respectively) (FIG. 5A). While concomitant administration of cipro at a dose of 1 mg/kg/day prevented further accumulation of bacteria within the liver and the spleen (Sandala et al., 2020. PLoS Pathog. 16(12): e1009192), mice still harbored a significant number of bacteria within these organs following treatment (FIG. 5B-C). In order to determine if the bacterial burden in these organs could be further reduced by increasing the concentration of cipro, we tested cipro doses of 2 and 4 mg/kg/day—either alone or in combination with 7d—over the same 10-day treatment period. Similar to treatment with 1 mg/kg/day cipro alone (Sandala et al, 2020. PLoS Pathog. 16(12): e1009192; Gonzalez et al., 2018. Sci Rep. 8(1): 222), treatment with 2 mg/kg/day cipro alone had no statistically significant effect on bacterial burden within the gallbladder of infected mice, though it did reduce the number of bacteria recovered from the liver and spleen (FIG. 5E-F). Further increasing dose of cipro administered in combination with 7d to 4 mg/kg/day further reduced bacterial burden in all organs such that the number of CFUs recovered neared the limit of detection (FIG. 5D-F). However, while treatment with 4 mg/kg/day cipro alone significantly reduced bacterial gallbladder burden by a factor of 3-4 logs (FIG. 5D), treatment with 7d and 4 mg/kg/day cipro resulted in an additional significant reduction of gallbladder bacteria.

### CONCLUSIONS

In conclusion, a structure-activity relationship study of compound 1 involving the halophenyl alkyl tail and the linker between the tail and the core was conducted. The initial library synthesized (FIG. 2) revealed that compounds with chlorine (4a) and bromine (4b) substitutions on the tail returned higher activity in terms of biofilm dispersion than compound 1. The subsequent libraries determined that the optimum length of the linker between the piperidine core and the tail was two methylene units (Table 2 and Table 3). Additionally, incorporation of chlorine in the 3-position resulted in an improved IC₅₀ of over two-fold (6a vs 4c) and in the 4-position of over four-fold (6b vs 4d), while bromine incorporation at these positions had a larger effect on dispersion activity (6b vs 6d). Three compounds (7b, 7d, and 8j) were selected for their superior IC₅₀ and EC₅₀ values and were tested alongside compound 1 in vivo in a murine model of gallbladder Salmonella chronic carriage with S. Typhimurium. These compounds dispersed Salmonella biofilms within the gallbladder to varying degrees, with compound 7d proving the most effective, reducing the bacterial burden in the gallbladder by 4.5-5 logs. Combination of compounds with ciprofloxacin effectively prevents the accumulation of bacteria released from the gallbladder to distal organs in an antibiotic dose-dependent manner. Further modifications of the lead compounds from this study are ongoing, as well as mechanism of action studies to determine the target of these molecules.

### Experimental Setup

All commercial solvents and reagents were purchased from VWR, Sigma-Aldrich, Oakwood Chemical, Matrix Scientific, or Enamine Ltd and used without any further purification. Reactions were monitored via thin layer chromatography (TLC) using glass-backed pre-coated silica gel plates from VWR (TLC Silica Gel 60 Sheets, MilliporeSigma, F254, 60 Å pore, 230-400 mesh) using UV visualization, ninhydrin stain, p-anisaldehyde stain, and/or vanillin stain as visualizing agent. Column chromatography was performed using silica gel (60 Å, particle size 40-60 μm, VWR). Solvent system for compound purification was a mixture of ammonia-saturated methanol in DCM with an initial DCM column flush unless otherwise indicated. Ammonia-saturated methanol was prepared by bubbling ammonia (Airgas) into methanol over the course of 15 minutes. Deuterated solvents for NMR characterization were purchased from MilliporeSigma via VWR and used as is, with the exception of chloroform-d, to which molecular sieves were added (4 Å, grade 514, mesh 8-12, Macron Fine Chemicals). All NMR spectra were performed at room temperature and recorded on either a Bruker AVANCE III HD 400 Nanobay spectrometer or a Bruker AVANCE III HD 500 without the use of signal suppression function and calibrated using the residual un-deuterated solvent peak (CDCl₃: δ 7.26 ppm ¹H NMR, 77.16 ppm ¹³C NMR; CD₃OD: δ 3.31 ppm ¹H NMR, 49.00 ppm ¹³C NMR). Proton (¹H) NMR are reported as follows: chemical shift in ppm (multiplicity, coupling constant(s) in Hz, relative integration). Abbreviations used are s, singlet; d, doublet; t, triplet; q, quartet; m, multiplet; br, broad. High resolution mass spectra (HRMS) were recorded on a Bruker micrOTOF II by electrospray ionization (ESI) time of flight (TOF) experiments using direct infusion in 9:1 acetonitrile:water. Analyses were performed by the mass spectrometry and proteomics facility at University of Notre Dame and reported as m/z. All compounds were characterized and tested at ≥95% purity as determined by liquid chromatography on either a Bruker micrOTOF-Q II by the University of Notre Dame mass spectrometry and proteomics facility or an Advion LC-MS 2020 with Kinetex, 2.6 mm, C18 50×2.10 mm.

General Synthetic Procedure for Carboxylic Acid Reduction. To an oven-dried 250 mL 3-necked round bottom flask was added 50 mL anhydrous tetrahydrofuran (THF) under argon and cooled to 0° C. LiAlH₄ (9.0 mmol, 9.0 mL 1M solution in THF) was added and allowed to cool. Carboxylic acid (3.0 mmol) was dissolved in 15 mL anhydrous THF and added dropwise to the solution over 30 minutes. Reaction was monitored by TLC and allowed to stir for an additional 1.5 hours. Reaction mixture was then quenched with a saturated solution of Rochelle's salt (20 mL) and allowed to stir for 5 minutes. Organic layer was removed and evaporated under reduced pressure. Crude oil was dissolved in ethyl acetate (100 mL), washed with brine (25 mL), dried over sodium sulfate, filtered, and concentrated in vacuo to yield an oil.

General Synthetic Procedure for Alcohol Mesylation. The alcohol (3.0 mmol), either purchased or crude reduced carboxylic acid, was dissolved in 50 mL anhydrous dichloromethane (DCM) and added to an oven-dried round bottom flask under argon. Triethylamine (6.0 mmol) was added and the solution was cooled to 0° C. Methanesulfonyl chloride (9.0 mmol) was added in one portion and the solution was allowed to stir for 30 minutes at 0° C. followed by 2 hours as it warmed to room temperature. The mixture was quenched with 20 mL di-H₂O and extracted with DCM (3×30 mL). Organic layers were combined and washed with brine, dried over sodium sulfate, and concentrated in vacuo. Crude mixture was then carried forward without further purification.

General Synthetic Procedure for Piperidine Alkylation. To an oven-dried two-necked 100 mL round bottom flask under argon was added anhydrous acetonitrile (30 mL) and tert-butyl methyl(piperidin-4-ylmethyl)carbamate (0.50 mmol). Oven-dried K₂CO₃ (1.5 mmol) was added to the flask in one portion, and the resulting mixture was heated to reflux. Mesylated intermediate or brominated starting material was added neat in one portion (1.25 mmol) and the reaction mixture was stirred under reflux for 24 hours. The reaction was then cooled and transferred to a single-necked 250 mL round bottom to be concentrated under reduced pressure. The crude was then dissolved in di-H₂O (50 mL) and extracted with DCM (3×30 mL). The organic layers were collected and washed with brine (30 mL), dried over anhydrous sodium sulfate, filtered, evaporated in vacuo and purified via flash column chromatography using a gradient of 2-5% methanol saturated with ammonia in DCM.

General Synthetic Procedure for Boc Deprotection. Boc-protected intermediate (0.40 mmol) was dissolved in 1 mL DCM. 2 mL trifluoroacetic acid (30 mmol) was added and the reaction was allowed to stir at room temperature open to atmosphere for 1 hour. Methanol (2 mL) was then added and mixture was evaporated in vacuo to dryness. Addition of methanol was repeated four times until no further vapors evolved upon addition of solvent. Crude intermediate was dried for 18 hours under vacuum and no further purification was performed.

General Synthetic Procedure for 3-(bromomethyl)thiophene Addition. Deprotected intermediate (0.40 mmol) was dissolved in 23 mL anhydrous acetonitrile and was transferred to an oven-dried 100 mL three-necked flask under argon. K₂CO₃ (1.50 mmol) was added and mixture was heated to reflux while stirring. 3-(bromomethyl)thiophene (0.45 mmol) was dissolved in 2 mL anhydrous acetonitrile and added to the reaction dropwise over one hour. The reaction was checked for completion by TLC after full addition of 3-(bromomethyl)thiophene, then cooled and transferred to a single-necked 250 mL round bottom flask and evaporated in vacuo. The crude was then dissolved in di-H₂O (50 mL) and extracted with DCM (3×30 mL). The organic layers were collected and washed with brine (30 mL), dried over anhydrous sodium sulfate, filtered, and evaporated under reduced pressure. The crude mixture was purified via column chromatography using 2-5% methanol saturated with ammonia in DCM.

General Synthetic Procedure for Making HCl Salts. Pure product was dissolved in 1 mL methanol and glacial hydrochloric acid (0.1 mL) was added to make a salt. The product was evaporated in vacuo. Methanol addition and subsequent evaporation was repeated six times until no further fumes evolved upon solvent addition. Solid obtained was dried under vacuum for 24-48 hours to yield the salt of the pure product.

Synthetic Procedure for Carboxylic Acid Reduction and Mesylation with Iodinated Compounds. To an oven-dried 2-necked round bottom flask under argon was added anhydrous THF (50 mL) and iodophenylacetic acid (1.9 mmol). The reaction mixture was cooled to 0° C. and NaBH₄ (5.7 mmol) was added in three portions and allowed to stir for 20 minutes. BF₃·Et₂O (3.8 mmol) was added via syringe pump over 15 minutes and the reaction was allowed to warm to room temperature and stirred for 16 hours. The reaction was quenched slowly with 10 mL cold methanol and evaporated in vacuo. The residue was dissolved in ethyl acetate (75 mL), washed with 1N HCl (50 mL), dried over sodium sulfate, filtered, and evaporated in vacuo. The intermediate was carried forward with no further purification. The general synthetic procedure for mesylation was followed to yield an oil.

Synthetic Procedure for Piperidine Alkylation with Iodinated Compounds. To an oven-dried two-necked 100 mL round bottom flask under argon was added anhydrous acetonitrile (30 mL) and tert-butyl methyl(piperidin-4-ylmethyl)carbamate (0.50 mmol). Oven-dried K₂CO₃ (1.5 mmol) was added to the flask in one portion, and the resulting mixture was heated to 55° C. Mesylated intermediate or brominated starting material was added neat in one portion (1.25 mmol) and the reaction mixture was stirred at 55° C. for 18 hours. The reaction was then cooled and transferred to a single-necked 250 mL round bottom to be concentrated under reduced pressure. The crude was then dissolved in di-H₂O (50 mL) and extracted with DCM (3×30 mL). The organic layers were collected and washed with brine (30 mL), dried over anhydrous sodium sulfate, filtered, evaporated in vacuo and purified via flash column chromatography using a gradient of 2-5% methanol saturated with ammonia in DCM. Subsequent Boc-deprotection of the product followed the general synthetic procedure for Boc-deprotection.

Synthetic Procedure for 3-(bromomethyl)thiophene Addition with Iodinated Compounds. Deprotected intermediate (0.40 mmol) was dissolved in 23 mL anhydrous acetonitrile and was transferred to an oven-dried 100 mL three-necked flask under argon. K₂CO₃ (1.50 mmol) was added and mixture was heated to 55° C. while stirring. 3-(Bromomethyl)thiophene (0.45 mmol) was dissolved in 2 mL anhydrous acetonitrile and added to the reaction dropwise over one hour. The reaction was checked for completion by TLC after full addition of 3-(bromomethyl)thiophene, then cooled and transferred to a single-necked 250 mL round bottom flask and evaporated in vacuo. The crude was then dissolved in di-H₂O (50 mL) and extracted with DCM (3×30 mL). The organic layers were collected and washed with brine (30 mL), dried over anhydrous sodium sulfate, filtered, and evaporated under reduced pressure. The crude mixture was purified via column chromatography using 2-5% methanol saturated with ammonia in DCM to obtain product. Subsequent creation of the HCl salt proceeded using the general synthetic procedure for making HCl salts.

Synthetic Procedure for Piperidine Alkylation with Starting Material Containing One or Three Methylene Linkers. To an oven-dried two-necked 100 mL round bottom flask under argon was added anhydrous acetonitrile (30 mL) and tert-butyl methyl(piperidin-4-ylmethyl)carbamate (0.50 mmol). Oven-dried K₂CO₃ (1.5 mmol) was added to the flask in one portion, and the resulting mixture was heated to reflux. Brominated starting material (0.75 mmol) was added neat in one portion and the reaction mixture was stirred at reflux for 14-18 hours, checking via TLC for completion. The reaction was then cooled, transferred to a single-necked 250 mL round bottom, and concentrated under reduced pressure. The crude was then dissolved in di-H₂O (50 mL) and extracted with DCM (3×30 mL). The organic layers were collected and washed with brine (30 mL), dried over anhydrous sodium sulfate, filtered, evaporated in vacuo and purified via flash column chromatography using a gradient of 2-5% methanol saturated with ammonia in DCM. Subsequent synthesis followed general procedures outlined above.

Salmonella biofilm growth and crystal violet staining. S. Typhimurium ATCC 14028 was streaked onto Luria-Bertani (LB) (Thermo Fisher Scientific, Catalog No #BP1426) agar plates and incubated at 37° C. overnight. For biofilm assays, individual bacterial colonies were picked and used to inoculate LB broth for overnight liquid cultures, which were grown at 37° C. with aeration using a rotating drum. Overnight cultures were normalized to an OD₆₀₀ of 0.8 (˜6.4×10⁸ colony forming units (CFU)/mL, further diluted 1:100 into minimal media (TSB (Thermo Fisher Scientific, Catalog No #DF0370-007-5) diluted 1:20 in ddH₂O), and added to non-treated, flat-bottom polystyrene 96-well plates (Corning, Kennebunkport, ME) at a volume of 100 L/well. Biofilm plates were incubated at 30° C. on a Fisherbrand™ nutating mixer (Thermo Fisher Scientific, Waltham, MA; 20° fixed angle, 24 rpm) for a total of 24 or 48 h for inhibition and dispersion assays, respectively. Biofilm growth was measured using a semi-quantitative method via CV staining. Biofilm plates were then submerged in dH₂O to wash away any remaining non-adherent bacteria and heat fixed (1h, 60° C.). Biofilms were then stained with a 33% crystal violet solution (6 mL PBS, 3.3 mL crystal violet, 333 μL methanol, 333 μL isopropanol) for 5 min. and washed twice by submerging in dH₂O before releasing the bound dye with 33% glacial acetic acid. Biofilm growth was then quantified by measuring the optical density of the solubilized dye at 570 nm using a spectrophotometer (Molecular Devices, SpectraMax M5).

Biofilm Inhibition and Dispersion Assays and IC₅₀/EC₅₀ Determination. For biofilm inhibition (IC₅₀) assays, S. Typhimurium biofilms were grown as described above but with various concentrations (0.2-100 μM) of compound (diluted in media from 100 mM stock solutions) or vehicle (DMSO) supplied in the media at the time of inoculation. Biofilms were then grown as described above for a total of 24 h prior to CV staining. For biofilm dispersion (EC₅₀) assays, S. Typhimurium biofilms were grown in media only for a total of 24 h as described above. Spent media was then removed and replaced with fresh media containing various concentrations (1.56-200 μM) of compound (diluted from 100 mM stock solutions) or vehicle. Biofilms were then incubated for an additional 24 h in the presence of compounds prior to CV staining. IC₅₀/EC₅₀ values were calculated by plotting normalized compound activity (percent biofilm formed and percent remaining, respectively) as a function of log₁₀ compound concentration and fitting a dose response curve (log[inhibitor] vs. normalized response, variable slope).

Murine model of chronic Salmonella gallbladder carriage and evaluation of compound therapeutic efficacy in combination with ciprofloxacin. A total of 46 adult 129X1/SvJ NRAMP^{+/+} mice (The Jackson Laboratory, Bar Harbour, ME) were used in this study. As described previously (Crawford et al., 2010. Proc Natl Acad Sci USA. 107(9): 4353-4358), mice were fed a lithogenic diet (conventional mouse chow supplemented with 1% cholesterol and 0.5% cholic acid; Envigo, Indianapolis, IN) for 8 weeks prior to infection in order to promote the formation of gallstones. Liquid cultures of S. Typhimurium ATCC 14028 were diluted in sterile PBS to a final inoculum density of ˜5×10³ CFU/mL, and mice were infected with ˜10³ CFU via injection of 200 μL inoculum into the intraperitoneal (I.P.) cavity. Mice were randomly assigned to treatment groups in two separate experiments. In the first experiment, treatment groups were as follows: vehicle (5% [v/v] DMSO in PBS), 5 mg/kg/day compound 1+1 mg/kg/day ciprofloxacin (cipro, Fluka cat. Number R1678), 5 mg/kg/day 7b+1 mg/kg/day cipro, 5 mg/kg/day 7d+1 mg/kg/day cipro, and 5 mg/kg/day 8j+1 mg/kg/day cipro. In the second experiment, treatment groups were: vehicle (same as above), 2 mg/kg/day cipro alone, 4 mg/kg/day cipro alone, 5 mg/kg/day 7d+2 mg/kg/day cipro, and 5 mg/kg/day 7d+4 mg/kg/day cipro. Treatments were administered daily via I.P. injection from days 5-15 post-infection. On day 15 post-infection, mice were euthanized and gallbladders, livers, and spleens were removed and homogenized in a volume of 1 mL sterile PBS using a TissueLyser LT bead mill (Qiagen, Valencia, CA). Tissue homogenates were serially diluted in PBS, plated onto LB agar, and incubated at 37° C. for 16h in order to quantify bacterial burden via CFU enumeration.

Statistical Information. For IC₅₀/EC₅₀ experiments, data represents the average ± the standard deviation for three biological replicates. In graphs of animal data, CFU values for individual animals were plotted as data points and treatment group averages are represented by horizontal lines; statistical analyses of CFU values were conducted using log-transformed values. All data transformations and statistical analyses were performed using GraphPad Prism 8, and p values<0.05 were considered significant unless otherwise specified (i.e. when correcting for multiple comparisons).

### Example 2: Variation of the Aromatic Group

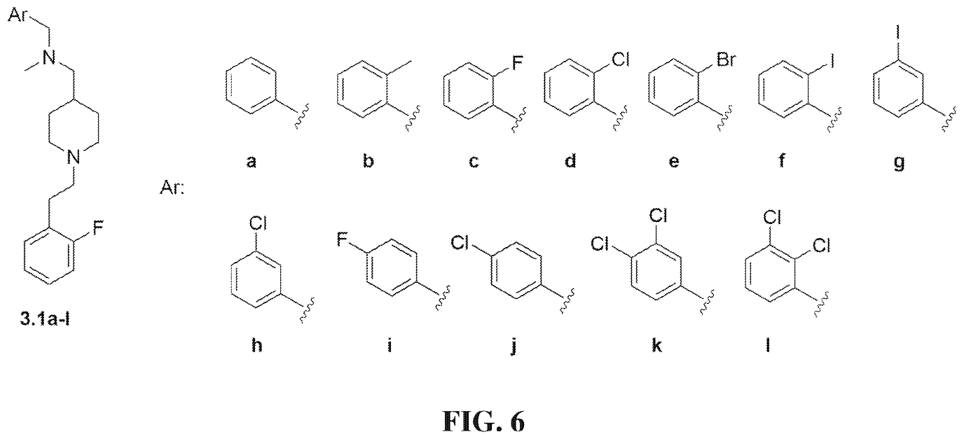

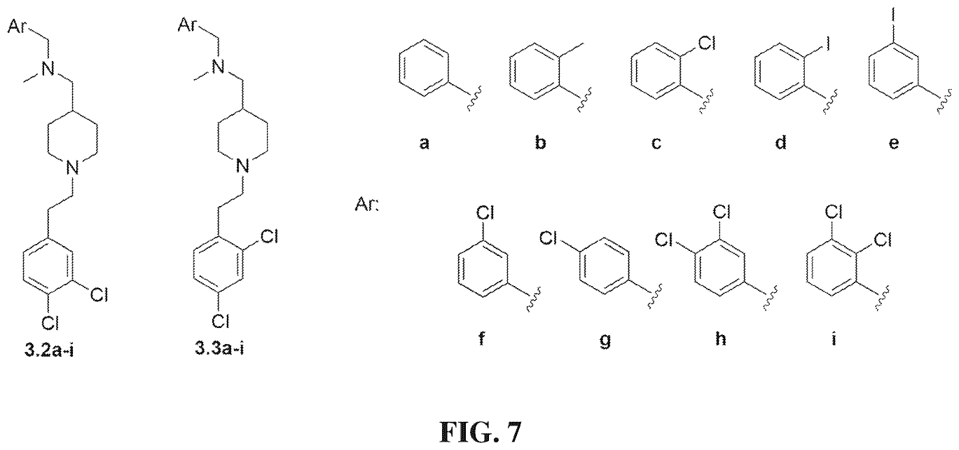

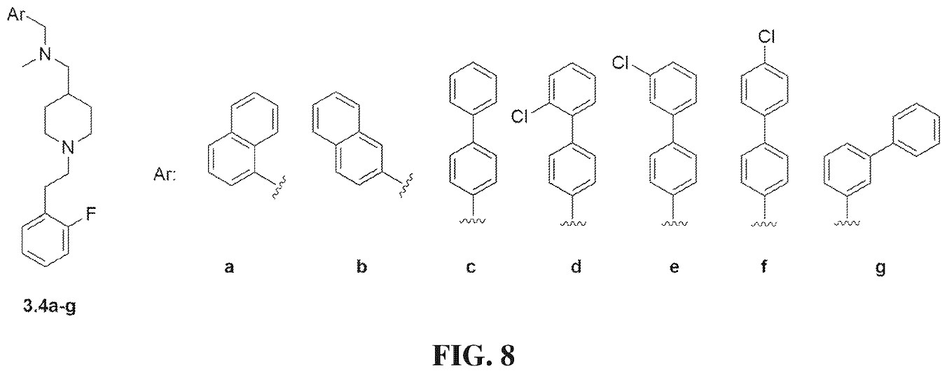

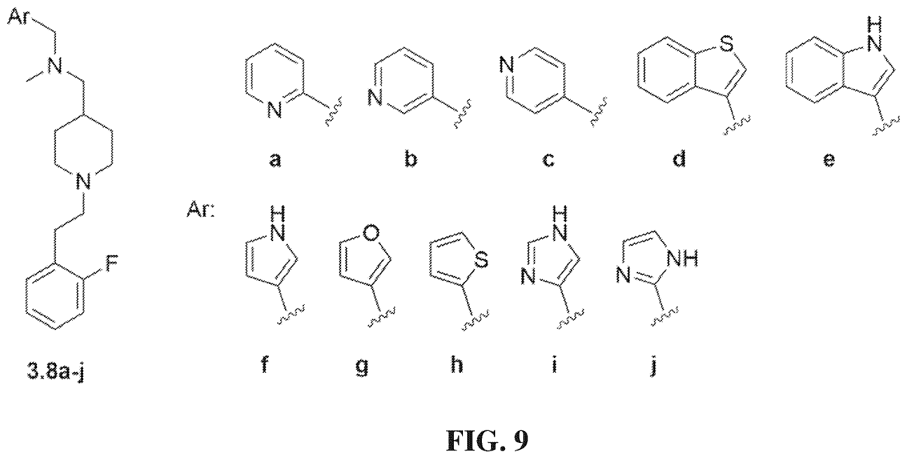

The inventors have prepared a number of biofilm inhibitors that included various different aromatic groups as the head group for the compounds. The compounds were synthesized and tested for activity using the methods described in Example 1. The compounds are shown in FIGS. 6-9, while the associated activity values for the compounds are shown in Tables 4-7 below. All values are in μM and are presented as the mean± of the standard deviation.

TABLE 4
IC50 AND EC50 VALUES FOR COMPOUNDS 3.1A-L

| Compound | IC₅₀ (μM) | EC₅₀ (μM) |
|---|---|---|
| 3.1a | 5.0 | >75 |
| 3.1b | 4.4^{a} | >75 |
| 3.1c | 14.9^{a} | >75 |
| 3.1d | 6.3^{b} | >75 |
| 3.1e | 6.5^{b} | 42 |
| 3.1f | 4.4^{b} | 53 |
| 3.1g | 1.7^{a} | 39 |
| 3.1h | 5.6^{a} | >75 |
| 3.1i | 5.5 | >75 |
| 3.1j | 0.54^{a} | 54 |
| 3.1k | 1.4^{b} | 41 |
| 3.1l | 5.6^{a} | 43 |

All values are in micromolar concentrations and, when available, are presented as the mean ± the standard deviation.
^{a}Growth inhibition at ≥100 μM.
^{b}Growth inhibition at ≥50 μM.

TABLE 5
IC50 AND EC50 VALUES FOR COMPOUNDS
3.2A-I, AND 3.3A-I.

| Compound | IC₅₀ (μM) | EC₅₀ (μM) |
|---|---|---|
| 3.2a | 0.59 | 35 |
| 3.2b | 2.2 | 28 |
| 3.2c | 3.7^{a} | 25 |
| 3.2d | 3.4^{a} | 26 |
| 3.2e | 1.4^{b} | 21 |
| 3.2f | 2.3^{a} | 21 |
| 3.2g | 1.2^{b} | 17 |
| 3.2h | 0.96^{c} | 17 |
| 3.2i | 2.5^{a} | 25 |
| 3.3a | 4.3 ± 2.1 | 11 ± 7 |
| 3.3b | TBD | TBD |
| 3.3c | 11.3 ± 7.3 | 14 ± 10 |
| 3.3d | 10.1 ± 4.5 | >50 |
| 3.3e | 5.4 ± 3.9 | 18 ± 15 |
| 3.3f | TBD | TBD |
| 3.3g | TBD | TBD |
| 3.3h | TBD | TBD |
| 3.3i | 6.5 ± 2.5 | 8 ± 4 |

All values are in micromolar concentrations and, when available, are presented as the mean ± the standard deviation.
^{a}Growth inhibition at ≥25 μM.
^{b}Growth inhibition at ≥12.5 μM.
^{c}Growth inhibition at ≥6.25 μM.
TBD: To be determined/not yet tested.

TABLE 6
IC50 AND EC50 VALUES FOR COMPOUNDS 3.4A-B.

| Compound | IC₅₀ (μM) | EC₅₀ (μM) |
|---|---|---|
| 3.4a | 4.2 ± 2.9 | 12 ± 8 |
| 3.4b | 0.74 ± 0.56 | 13 ± 9 |

TABLE 7
IC50 AND EC50 VALUES FOR COMPOUNDS 3.8A-C.

| Compound | IC₅₀ (μM) | EC₅₀ (μM) |
|---|---|---|
| 3.8a | >20 | >50 |
| 3.8b | >20 | >50 |
| 3.8c | >20 | >50 |

Compounds NDM-29 AND NDM-30 were also prepared and tested.

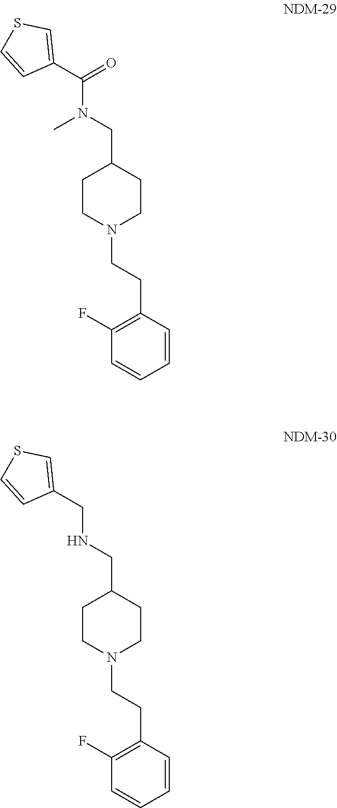 [SMILES: CN(CC1CCN(CCc2ccccc2F)CC1)C(=O)c1ccsc1 ; Fc1ccccc1CCN1CCC(CNCc2ccsc2)CC1]

| Compound | IC₅₀ (μM) | EC₅₀ (μM) |
|---|---|---|
| NDM-29 | >20 | >75 |
| NDM-30 | 12 | >75 |

The complete disclosure of all patents, patent applications, and publications, and electronically available materials cited herein are incorporated by reference. The foregoing detailed description and examples have been given for clarity of understanding only. No unnecessary limitations are to be understood therefrom. In particular, while various theories are presented describing possible mechanisms through with the compounds are effective, the compounds are effective regardless of the particular mechanism employed and the inventors are therefore not bound by theories described herein. The invention is not limited to the exact details shown and described, for variations obvious to one skilled in the art will be included within the invention defined by the claims.

1. A compound according to formula I: 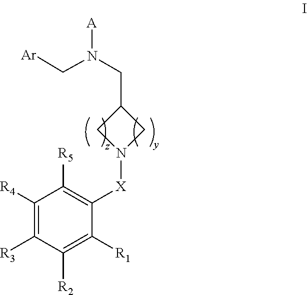
wherein Ar is an aryl or heteroaryl group, A is a C₁-C₃ alkyl, Z and Y are independently C₁-C₃ alkylene, X is C₁-C₃ alkylene, and R¹-R⁵ are selected from —H, halogen, C₁-C₃ alkyl, or phenyl, or a pharmaceutically acceptable salt thereof.

2. The compound of claim 1, wherein one or two of R¹-R⁵ are selected from bromine, chlorine, iodine, or phenyl, and the remainder of R¹-R⁵ are hydrogen.

3. The compound of claim 1, wherein Ar is a thiophene group.

4. The compound of claim 1, wherein Ar is halogenated phenyl group.

5. The compound of claim 1, wherein X is C₂ alkylene.

6. The compound of claim 1, wherein Z and Y are both C₂ alkylene.

7. The compound of claim 1, wherein two of R¹-R⁵ are chlorine, bromine, or iodine, and the remaining R groups are —H.

8. The compound of claim 1, wherein R¹ and R² are chlorine, and R³—R⁵ are —H.

9. The compound of claim 1, wherein R¹ and R³ are chlorine, and R², R⁴, and R⁵ are —H.

10. The compound of claim 1, wherein R² and R³ are bromine, and R¹, R⁴, and R⁵ are —H.

11. The compound of claim 1, wherein R³ is phenyl, and R¹, R², R⁴, and R⁵ are —H.

12. A method of decreasing bacterial biofilm in a subject, comprising administering a therapeutically effective amount of a compound according to formula I: 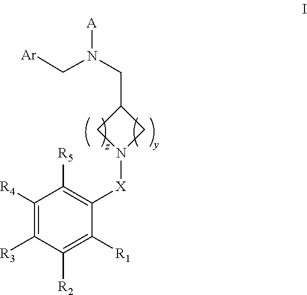
to the subject; wherein Ar is an aryl or heteroaryl group, A is a C₁-C₃alkyl, Z and Y are independently C₁-C₃alkylene, X is C₁-C₃ alkylene, and R¹-R⁵ are selected from —H, halogen, C₁-C₃ alkyl, or phenyl, or a pharmaceutically acceptable salt thereof

13. The method of claim 12, wherein one or two of R¹-R⁵ are selected from bromine, chlorine, iodine, or phenyl, and the remainder of R¹-R⁵ are hydrogen.

14. The method of claim 12, wherein X is C₂ alkylene.

15. The method of claim 12, wherein the compound is administered together with a pharmaceutically acceptable carrier.

16. The method of claim 12, wherein the subject is human.

17. The method of claim 12, wherein the biofilm is formed by Salmonella.

18. The method of claim 12, wherein two of R¹-R⁵ are chlorine, bromine, or iodine, and the remaining R groups are —H.

19. The method of claim 12, wherein the method comprises decreasing bacterial biofilm in the gallbladder of the subject.

20. A method of treating or preventing bacterial infection in a subject in need thereof, comprising administering a therapeutically effective amount of an antibiotic and a compound according to formula I: 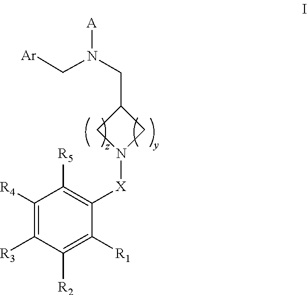
to the subject; wherein Ar is an aryl or heteroaryl group, A is a C₁-C₃ alkyl, Z and Y are independently C₁-C₃ alkylene, X is C₁-C₃ alkylene, and R¹-R⁵ are selected from —H, halogen, C₁-C₃ alkyl, or phenyl, or a pharmaceutically acceptable salt thereof.

21. The method of claim 20, wherein the antibiotic is ciprofloxacin.
